(19) United States
(12) Patent Application Publication
(10) Pub. No.: US 2002/0044937 A1
(43) Pub. Date: Apr. 18, 2002

(54) PROTEIN-POLYCATION CONJUGATES

(76) Inventors: MAX L. BIRNSTIEL, WIEN (AT); MATTHEW COTTEN, WIEN (AT); ERNST WAGNER, LANGENZERSDORF (AT)

Correspondence Address: STERNE KESSLER GOLDSTEIN & FOX, SUITE 600, 1100 NEW YORK AVENUE NW, WASHINGTON, DC, 200053934

(21) Appl. No.: 08/380,200

(22) Filed: Jan. 30, 1995

Related U.S. Application Data
(63) Continuation of application No. 07/946,498, filed on Nov. 9, 1992, now abandoned.

(30) Foreign Application Priority Data
May 10, 1991 (US) PCT/EP91/00875
May 18, 1990 (AT) A 1110/90
Mar. 29, 1991 (DE) P 41 10 410.2

(51) Int. Cl. A61K 39/395; A61K 39/44; A61K 39/40; A61K 39/42; C07K 16/00; C07K 17/00; C07K 17/14; C12P 21/08

(52) U.S. Cl. 424/178.1; 424/182.1; 530/391.1; 530/391.7

(57) ABSTRACT

New protein-polycation conjugates are capable of forming soluble complexes with nucleic acids or nucleic analogs. The protein portion of these conjugates is a protein capable of linking with a cellular surface protein expressed by cells of the T-cell lineage, so that the complexes thus obtained are absorbed by cells which express the T-cell surface protein. Complexes useful in pharmaceutical compositions contain a therapeutically or gene therapeutically active nucleic acid.

The invention relates to new protein-polycation conjugates for transporting nucleic acids into human or animal cells.

In recent years, nucleic acids have acquired greater significance as therapeutically active substances.

Antisense RNAs and DNAs have proved to be effective agents for selectively inhibiting certain genetic sequences. Their mode of activity enables them to be used as therapeutic agents for blocking the expression of certain genes (such as deregulated oncogenes or viral genes) in vivo. It has already been shown that short antisense oligonucleotides can be imported into cells and perform their inhibiting activity therein (Zamecnik et al., 1986), even though the intracellular concentration thereof is low, partly because of their restricted uptake through the cell membrane owing to the strong negative charge of the nucleic acids.

Another method of selectively inhibiting genes consists in the application of ribozymes. Here again there is the need to guarantee the highest possible concentration of active ribozymes in the cell, for which transportation into the cell is one of the limiting factors.

Numerous solutions have already been proposed for improving the transportation of nucleic acids into living cells, which is one of the limiting factors in the therapeutic use thereof.

One of these possible solutions consists in directly modifying the nucleic acids, e.g. by substituting the charged phosphodiester groups with uncharged groups. Another possible method of direct modification consists in using nucleoside analogues.

Although some of these proposals represent a theoretically promising approach to solving the problem, they do have various disadvantages, e.g. reducing binding to the target molecule, a poorer inhibitory effect and possible toxicity.

An alternative approach to the direct modification of the oligonucleotides consists in leaving the oligonucleotide per se unchanged and providing it with a group which gives it the desired properties, e.g. with molecules which facilitate transportation into the cells.

In addition to inhibiting genes there is also a need for an efficient system for introducing nucleic acid into living cells in gene therapy. For this, genes are locked into cells in order to achieve the synthesis of therapeutically active genetic products in vivo, e.g. to replace the defective gene in cases of genetic defect. Examples of possible uses in genetically caused diseases in which gene therapy constitutes a promising approach are haemophilia, beta-thalassaemia and “Severe Combined Immune Deficiency” (SCID), a syndrome caused by a genetically induced lack of the enzyme adenosine deaminase. The “conventional” gene therapy is based on the principle of achieving a permanent cure by a single treatment. However, there is also a need for methods of treatment in which the therapeutically effective DNA (or mRNA) is administered like a drug (“gene therapeutic agent”) either once or repeatedly, as required. Possible applications for this principle are in immune regulation in which a humoral or intracellular immunity is achieved by the administration of functional nucleic acid which codes for a secreted protein antigen or for a non-secreted protein antigen, by means of an inoculation. Examples of genetic defects in which a nucleic acid coding for the defective gene can be administered, in a form tailored to the individual requirements, include muscular dystrophy (dystrophin gene), cystic fibrosis (transmembrane regulator gene) and hypercholesterolaemia (HDL receptor gene). Methods of treatment by gene therapy are also of potential significance where hormones, growth factors or proteins with a cytotoxic or immunomodulating activity are to be synthesised in the body.

The technologies which have hitherto progressed furthest for the use of nucleic acids in gene therapy make use of retroviral systems for the transfer of genes into the cell (Wilson et al., 1990, Kasid et al., 1990). The use of retroviruses does, however, present problems because it involves, at least in a small percentage, the danger of side effects such as infection with the virus (by recombination with endogenous viruses and possible subsequent mutation into the pathogenic form) or by formation of cancer. Moreover, the stable transformation of the somatic cells of the patient as achieved by means of retroviruses is not desirable in every case since it may only make the treatment more difficult to reverse, e.g. if side effects occur.

There has therefore been a search for alternative methods of enabling the expression of non-replicating DNA in the cells.

There are various known techniques for the genetic transformation of mammalian cells in vitro, but their use in vivo is restricted (they include the introduction of DNA by means of liposomes, electroporation, microinjection, cell fusion, DEAE-dextran or the calcium phosphate precipitation method).

Recent efforts to develop methods for in vivo gene transfer have concentrated on the use of the cationic lipid reagent lipofectin; a plasmid injected by means of this reagent has been shown to be capable of being expressed in the body (Nabel et al., 1990).

Another recently developed method uses microparticles of tungsten or gold onto which DNA has been absorbed, by means of which the cells can be bombarded with high energy (Johnston, 1990, Yang et al., 1990). Expression of the DNA has been demonstrated in various tissues.

A soluble system which can be used in vivo to convey the DNA into the cells in targeted manner was developed for hepatocytes and is based on the principle of coupling polylysine to a glycoprotein to which a receptor provided on the hepatocyte surface responds and then, by adding DNA, forming a soluble glycoprotein/polylysine/DNA complex which is absorbed into the cell and, once absorbed, allows the DNA sequence to be expressed (G. Y. Wu, C. H. Wu, 1987).

This system is specific to hepatocytes and is defined, in terms of its function, by the relatively well characterised absorption mechanism by means of the asialoglycoprotein receptor.

A broadly applicable and efficient transport system makes use of the transferrin receptor for absorbing nucleic acids into the cell by means of transferrin-polycation conjugates. This system is the subject of European Patent Application A1 388 758. It was shown that transferrin-polycation/DNA complexes are efficiently absorbed and internalised in living cells, using as the polycation component of the complexes polylysine of various degrees of polymerisation and protamine. Using this system, inter alia, a ribozyme gene inhibiting the erbB-oncogene was introduced into erbB-transformed hen cells and the erbB inhibiting effect was demonstrated.

The aim of the present invention was to provide a system by means of which it would be possible to transport nucleic acids selectively into higher eukaryotic cells, particularly cells of the T-cell lineage. (For the sake of simplicity cells of the T-cell lineage will hereinafter be referred to as T-cells. This term includes precursor T-cells and the lines diversifying from them, including the mature T-cells).

T-lymphocytes (T-cells) differentiate in the thymus. One of their functions is to support the B-cells in the antigen response. One of the characteristics of T-cells is that they do not recognise free antigen but only fragments of antigens. T-cells recognise a peptide-antigen fragment of this kind on the surface of target cells by means of a T-cell antigen receptor (TCR) which interacts with an antigen bound to an MHC (major histocompatibility complex) molecule. The specific antigen recognition requires the cooperation of another receptor, CD4 or CD8, with non-polymorphous regions of MHC. This interaction of TCR and either CD4 or CD8 with an MHC molecule on target cells is necessary for the formation of the specific capabilities of T-cells during the thymic development and enables the antigen-specific activation of mature T-cells. T-cells which recognise antigen associated with Class I MHC molecules (predominantly killer cells), express CD8; cells which recognise Class II associated antigens (predominantly helper cells) express CD4. The tasks of CD4⁺ cells within the scope of the immune response are, as well as inducing the B-cell function, to activate macrophages, secrete growth and differentiation factors for lymphoid cells, secrete factors which induce non-lymphoid cell functions, and to induce the suppressor, NK and cytotoxic T-cell function (Fauci, 1988).

In addition to its important role in immune recognition, CD4, a glycoprotein with a molecular weight of 55,000 which is present not only on T-cells but also, to a lesser extent, on monocytes/macrophages, plays a crucial role in infection with the HIV virus by acting as a receptor for the virus. HIV is the pathogen of AIDS (acquired immunodeficiency syndrome), a serious disease which is accompanied by progressive and irreversible damage to the immune system. This is caused in particular by a selective reduction in CD4⁺-T-cells.

Since its discovery the HIV virus has been investigated thoroughly in terms of its molecular biology, infectiousness and mechanisms of pathogenesis.

HIV is an RNA retrovirus which was originally called HTLV-III, LAV or ARV. The virus formerly known as HIV is nowadays frequently known as HIV 1 to distinguish it from a virus (HIV-2) detected in West African patients which is related to the SIV-virus and causes a syndrome which is indistinguishable from AIDS.

The HIV virus genome is well characterised. It is about 10 kb long and comprises the flanking LTR (long terminal repeat) sequences which contain regulatory sequences for replication as well as at least nine genes. These genes comprise not only the gag, pol and env genes common to all replicable retroviruses but also genes which are involved in maturation and morphogenesis (vpu and vif), genes which are involved in the regulation of virus replication (tat, rev and nef) and one gene of unknown function (vpr). The tat gene plays an important part in the amplification of virus replication by coding for a protein with a trans-activator function for HIV gene expression.

After binding to the CD4 molecule, which is a receptor having a high affinity for HIV (Dalgleish et al., 1984; Klatzmann et al., 1984; McDougal et al., 1986), the virus is absorbed into the cell and freed from its coat. There are conflicting views on this process. It has been proposed, inter alia, that in this process receptor-mediated endocytosis is involved (Maddon et al., 1986; Pauza and Price, 1988). However, this is contradicted by the observation that in order for the virus to enter the cell it is necessary for the transmembranal part (gp41) of the virus coat to fuse with the cell membrane, irrespective of the pH (Stein et al., 1987). It has also been observed that mouse cells which express human CD4 cannot be productively infected in spite of the binding of the virus to the cell. This result can be interpreted as showing that, as well as CD4, other proteins on human CD4⁺ cells may also be responsible for the internalisation of the virus.

The HIV virus is bound to the CD4 molecule by means of the virus coat protein (env).

The primary product of the env gene, gp160, is a precursor the cleaving of which (during maturation on the way through the ER and Golgi apparatus) yields the virion proteins gp120 and gp41. The cleaving of gp160 is necessary for the fusion of the virus with the cell and for infectiousness. gp120 is the outer coat glycoprotein and is present on the outside of the membrane of infected cells and virus particles. It has no membrane anchoring domain and remains attached to the membrane solely by non-covalent binding to gp41. gp120 contains the essential determinant for the binding of the virus to the receptor. The high-affinity bond between the virus and the cell membrane is achieved by interaction between a section of 40 amino acids at the C-terminus of gp120 and a domain close to the N-terminus of CD4. Although there are substantial differences in the gp120 sequences between the different HIV strains, the CD4 binding domains between HIV-1, HIV-2 and the related SIV viruses are conserved. Proteolytic fragments of 95 and 25 kDa have been isolated which are clearly domain-like subdivisions of gp120 and are capable of binding to CD4 in the same way as the original gp120 (Nygren et al., 1988).

There are various theories as to the course of the fusion process; however, there is no dispute as to the key role of gp41 in this process. gp41 has a hydrophobic sequence which is strongly homologous with fusion sequences at the N-terminus of transmembrane proteins of other viruses. It has been observed that the env proteins form an oligomer, whilst possibly an allosteric rearrangement of the oligomer on the virus membrane promotes the introduction of the gp41-N-terminus into the target cell membrane and also promotes fusion (the same effect is thought to be responsible for the formation of syncytia between infected cells).

One of the most promising approaches to the problem of blocking HIV infection appears to be neutralisation by a soluble, secreted form of the CD4 antigen (Smith et al.; 1987) which competes for binding to gp120.

A therapeutic possibility, once the body has been infected with HIV, of protecting the as yet uninfected cells or preventing activation of the latent virus in the affected cells., consists in administering nucleic acid molecules which inhibit virus replication.

For therapeutic applications of nucleic acids of this kind it is essential that they should be absorbed efficiently into the cell.

Within the scope of the present invention it has surprisingly been found that proteins which bind to a cell surface protein expressed by T-cells can be used to transport nucleic acids into cells which express the cell surface protein, if they are conjugated with polycations.

It has been found that the receptor used by the HIV virus during infection, namely CD4, can be used to transport nucleic acid into the cell by complexing the nucleic acid which is to be imported with a protein-polycation conjugate the protein content of which is a protein capable of binding to CD4, and bringing CD4 expressing cells into contact with the resulting protein-polycation/DNA complexes.

It has also been shown that, by means of antibody-polycation conjugates containing an antibody against CD7, DNA is introduced into cells of the T-cell lineage and expressed in these cells. (CD7 is a cell surface protein with an as yet unknown physiological role which has been detected on thymocytes and mature T-cells. CD7 is a reliable marker for acute T-cell leukaemia (Aruffo and Seed, 1987)).

Within the scope of the present invention it has thus been demonstrated, by means of protein-polycation conjugates with various proteins, all sharing the ability to bind to T-cell surface proteins, that the internalisation and expression of DNA can be carried out with the aid of such conjugates in cells which express the T-cell surface antigen in question.

The invention thus relates to new protein-polycation conjugates which are capable of forming complexes with nucleic acids, the protein content being a protein which is capable of binding to a cell surface protein expressed by T-cells, so that the complexes formed can be absorbed into cells which express the cell surface protein, especially T-cells.

In the description that follows, proteins or fragments thereof which are capable of binding to cell surface proteins of T-cells are referred to as T-cell binding proteins (TCBPs).

Examples of proteins capable of binding to CD4 (or CD7) are referred to as “CD4 (CD7) binding protein” or “CD4BP (CD7BP)”.

The invention further relates to TCBP-polycation/nucleic acid complexes in which the conjugates according to the invention are complexed with a nucleic acid which is to be transported into the target cells expressing the T-cell surface antigen to which the TCBP binds.

Within the framework of the invention it has been demonstrated that DNA in the form of the complexes according to the invention is efficiently absorbed into cells which express the particular T-cell surface antigen to which the TCBP binds, and the DNA is expressed therein, the uptake of DNA into the cell increasing as the conjugate content increases.

If antibodies are to be used as TCBPs, it is possible to use any antibody, particularly a monoclonal antibody, against a T-cell surface protein or fragment thereof which binds to the cell surface protein in question, e.g. Fab′ fragments (Pelchen-Matthews et al., 1989).

These include anti-CD4 antibodies which have a gp120 epitope which competes with the virus for binding to this epitope.

Instead of conventional monoclonal antibodies or fragments thereof it is possible to use antibody sections consisting of a combination of segments of the heavy and light chain or possibly of the heavy chain on its own. The preparation of such “alternative” antibodies by cloning by means of polymerase chain reaction and expression in E.coli have been briefly described (Sastry et al., 1989; Orlandi et al., 1989; Chaudhary et al., 1990).

As CD4BPs it is also possible to use HIV-1-gp120 or homologous proteins of related retroviruses or fragments thereof. gp120 fragments which are suitable for use within the scope of the present invention are those which are capable of binding to CD4 (Lasky et al., 1987), e.g. the 95-kDa and 25-kDa fragments, which have been shown to bind to CD4 (Nygren et al.; 1988). Such fragments may, for example, be obtained either by first preparing the entire gp120 protein by the recombinant method and subsequently carrying out proteolytic cleaving or, alternatively, to prepare the fragments themselves by the recombinant method.

The choice of TCBP is determined particularly by the target cells, e.g. by certain surface antigens or receptors which are specific or largely specific to one type of cell and thus enable a directed introduction of nucleic acid into this type of cell.

Depending on the surface antigen to which the protein contained in the conjugate binds, the conjugates according to the invention enable narrower or wider selectivity with regard to the cells which express T-cell surface protein and which are to be treated with nucleic acid and hence flexible use of nucleic acid which is therapeutically active or active in gene therapy.

Within the scope of the invention, it is convenient to use, as conjugate components, TCBPs which bind to the cell, with the result that the conjugate/DNA complexes are internalised, particularly by endocytosis, or TCBPs the binding/internalisation of which is carried out by fusion with cell membrane elements.

What is essential for the suitability of TCBPs within the scope of the invention is that

a) they should be recognised by the specific type of cell into which the nucleic acid is to be introduced and that their binding capacity is unaffected or not substantially affected if they are conjugated with the polycation, and

b) that within the scope of this property they are capable of carrying nucleic acid “piggyback” into the cell by the route which they use.

Provided that they meet the conditions defined in a) and b), basically all proteins which bind to T-cell surface antigens/receptors are suitable for use according to the present invention. These include antibodies against T-cell surface proteins which are specifically expressed on one or more examples of cells of a particular state of differentiation, e.g. antibodies against CD4, CD44, CD7, CD3, CD8 and the corresponding antibody fragments.

For targeted use on tumour cells it is particularly suitable to use antibodies against cell surface proteins specifically expressed on T-cells, so-called tumour markers, e.g. CD7.

In addition to antibodies and gp120 (fragments) it is possible to use, for the purposes of the invention, all natural antigens which satisfy the requirements mentioned under a) and b).

Polycations which are suitable according to the invention include, for example, homologous polycations such as polylysine, polyarginine, polyornithine or heterologous polycations having two or more different positively charged amino acids, these polycations possibly having different chain lengths, as well as non-peptide synthetic polycations such as polyethyleneimine. Other suitable polycations are natural DNA-binding proteins of a polycationic nature such as histones or protamines or analogues or fragments thereof.

The following compounds may be used as polycations or (poly)peptides of a polycationic nature:

a) Protamines: These are small (MW up to about 8000) strongly basic proteins the positively charged amino acid groups of which (especially arginines) are usually arranged in groups and which neutralise the negative charges of nucleic acids by virtue of their polycationic nature (Warrant et al., 1978). The proteins which may be used within the scope of the present invention may be of natural origin or prepared by the recombinant method, whilst multiple copies may be prepared or modifications may be made in terms of molecular size and amino acid sequence. Corresponding compounds may also be chemically synthesised. A synthetic protamine may, for example, be synthesised by replacing amino acid groups which, in the natural protamine, have functions which are undesirable for the transporting function (e.g. condensation of DNA) with other suitable amino acids, and/or providing at one end an amino acid (e.g. cysteine) which enables the desired conjugation with CD4BP.

b) Histones: These are small DNA-binding proteins present in the chromatin containing a high proportion of positively charged amino acids (lysine and arginine) which enables them to bind to DNA independently of the nucleotide sequence and folding them into nucleosomes, the arginine-rich histones H3 and H4 being particularly suitable (Felsenfeld, 1978). With regard to the production and modifications the remarks made above for protamines apply.

c) Synthetic polypeptides such as homologous polypeptides (polylysine, polyarginine) or heterologous polypeptides (consisting of two or more examples of positively charged amino acids).

d) Non-peptide cations such as polyethyleneimines.

The size of the polycations is preferably chosen so that the sum of the positive charges is about 20 to 500, in accordance with the particular nucleic acid to be transported.

The TCBP-polycation conjugates according to the invention may be prepared chemically in a method known for the coupling of peptides, and if necessary the individual components may be provided before the coupling reaction with linker substances (this measure is necessary if there is no available functional group suitable for coupling such as a mercapto or alcohol group. The linker substances are bifunctional compounds which are reacted first with functional groups of the individual components, after which the modified individual components are coupled.

Depending on the desired properties of the conjugates, particularly with respect to their stability, coupling may be carried out by

a) Disulphide bridges which can be cleaved again under reducing conditions (e.g. using succinimidyl-pyridyldithiopropionate (Jung et al., 1981).

b) Using compounds which are largely stable under biological conditions (e.g. thioethers by reacting maleimido linkers with sulfhydryl groups of the linker bound to the second component).

c) Bridges which are unstable under biological conditions, e.g. ester bonds, or acetal or ketal bonds which are unstable under slightly acidic conditions.

It is also possible to prepare the conjugates according to the invention by the recombinant method, the advantage of this being that precisely defined and uniform compounds can be obtained, whereas chemical coupling produces conjugate mixtures which have to be separated.

The recombinant preparation of the conjugates according to the invention may be carried out using methods known for the preparation of chimeric polypeptides. The polycationic peptides may vary in their size and amino acid sequence. Production by genetic engineering also has the advantage of allowing modification of the TCBP part of the conjugate, for example by increasing the ability to bind to the cell surface protein, by suitable mutation, or by using a TCBP component which has been shortened to that part of the molecule which is responsible for binding to the cell surface protein (e.g. using gp120 fragments or “alternative” antibodies). It is particularly appropriate for recombinant production of the conjugates according to the invention to use a vector which contains the sequence coding for the TCBP component, as well as a polylinker into which the required sequence coding for the polycationic peptide has been inserted. In this way it is possible to obtain a set of expression plasmids from which the plasmid containing the desired sequence can be selected to be used as necessary for the expression of the conjugate according to the invention.

The molar ratio of TCBP to polycation is preferably 10:1 to 1:10, although it should be borne in mind that aggregates may be formed. However, this ratio may be within wide limits if necessary, provided that it satisfies the condition that complexing with the nucleic acid or acids to be transported into the cells takes place and provided that the complex formed is assured of being bound to the cell surface protein and conveyed into the cell. This can be checked by simple tests carried out in each individual case, e.g. by bringing cell lines which express the T-cell surface antigen into contact with the complexes according to the invention and then investigating them for the presence of nucleic acid or the gene product in the cell, e.g. by Southern blot analysis, hybridisation with radioactively labelled complementary nucleic acid molecules, by amplification using PCR or by detecting the gene product of a reporter gene.

The particular ratio selected will depend particularly on the size of the polycation molecule and the number and distribution of the positively charged groupings, criteria which are adapted to the size, structure and possible modifications of the nucleic acid or acids to be transported. The polycations may be identical or different.

For specific applications when using antibodies as TCBPs, particularly for screening in order to find suitable antibodies, it may be advantageous not to couple the antibody directly to the polycation: for efficient chemical coupling it is generally necessary to use a larger amount (more than 1 mg) of starting antibody and furthermore the coupling may optionally deactivate the antibody binding domain. To get round this problem and allow rapid screening of suitable antibodies it is first of all possible to prepare a protein A polycation conjugate to which the antibody is subsequently bound, optionally in a form already complexed with nucleic acid, just before the transfection of the cells, by means of the F_{c}-binding domain of protein A (Surolia et al., 1982). The nucleic acid complexes formed with the protein A conjugates allow rapid testing of antibodies for their suitability for importing nucleic acid into the particular type of cells to be treated. The coupling of protein A with the relevant polycation is carried out analogously to the direct coupling with the antibody. When protein A-antibody-polycation conjugates are used it may be advantageous first to incubate the cells which are to be treated with the antibody, to free the cells from excess antibody and then treat them with the protein A-polycation/nucleic acid complex. The protein A conjugates may be prepared by the recombinant method, depending on the polycation used.

The nucleic acids to be transported into the cell may be DNAs or RNAs, there being no restrictions on the nucleotide sequence. The term “nucleic acids” for the purposes of the present invention also includes modified nucleic acids provided that the modification does not affect the polyanionic nature of the nucleic acids and their complexing with the conjugates according to the invention; these modifications include, for example, the substitution of the phosphodiester group by phosphorothioates or the use of nucleoside analogues. Such modifications are common to those skilled in the art; a summary of nucleic acids modified in representative manners and generally referred to as nucleic acid analogues and the principle of action thereof are described in the article by Zon (1988).

With regard to the size of the nucleic acids the invention also allows a wide range. There is no theoretical upper limit imposed by the conjugates according to the invention, provided that the TCBP-polycation/nucleic acid complexes are assured of being conveyed into the cells. Any lower limit is a result of reasons specific to the particular application e.g. because antisense oligonucleotides of less than about 10 nucleotides cannot be used on the grounds of insufficient specificity. Using the conjugates according to the invention plasmids can also be conveyed into the cells.

It is also possible to convey different nucleic acids into the cells at the same time by means of the conjugates according to the invention.

Examples of suitable nucleic acids are the antisense oligonucleotides mentioned above or ribozymes with a virus-inhibiting effect on the grounds of complementarity to the gene sections essential for virus replication.

The preferred nucleic acid component of the TCBP-polycation-nucleic acid complexes according to the invention having an inhibiting effect on the grounds of complementarity is antisense DNA, antisense RNA or a ribozyme or the gene coding therefor. When using ribozymes and antisense RNAs it is particularly advantageous to use the genes coding therefor, optionally together with a carrier gene. By introducing the gene into the cell a considerable amplification of the RNA is achieved, compared with the introduction of RNA as such, and consequently a supply which is sufficient for the intended inhibition of biological reaction is assured. Particularly suitable carrier genes are the transcription units required for transcription by polymerase III, e.g. tRNA genes. Ribozyme genes, for example, may be inserted into them in such a way that when transcription is carried out the ribozyme is part of a compact polymerase III transcript. Suitable genetic units containing a ribozyme gene and a carrier gene transcribed by polymerase III are disclosed in European Patent Application A1 0 387 775. With the aid of the transport system according to the present invention the effect of these genetic units can be intensified, by ensuring an increased initial concentration of the gene in the cell.

In principle all sequences of the HIV gene the blocking of which causes the inhibition of viral replication and expression are suitable as target sequences for the construction of complementary antisense oligonucleotides or ribozymes or the genes coding therefor which can be used in the treatment of AIDS. Target sequences of primary importance are the sequences with a regulatory function, particularly of the tat-, rev- or nef-gene. Other suitable sequences are the initiation, polyadenylation, splicing tRNA primer binding site (PBS) of the LTR sequence or the tar-sequence.

Apart from nucleic acid molecules which inhibit as a result of being complementary to viral genes, it is also possible to use genes with a different mechanism of activity, e.g. those which code for virus proteins containing so-called transdominant mutations (Herskowitz, 1987). The expression of the gene products in the cell results in proteins which, in their function, dominate the corresponding wild type virus protein, as a result of which the latter cannot perform its usual function for virus replication and the virus replication is effectively inhibited. Basically, transdominant mutants of virus proteins which are necessary for replication and expression, e.g. gag-, tat- and rev-mutants, which have been shown to have an inhibiting effect on HIV-replication (Trono et al., 1989; Green et al., 1989; Malim et al., 1989) are suitable.

Other examples of therapeutically active nucleic acids are those with an inhibitory effect on oncogenes.

With the aid of the present invention it is also possible to transport genes or sections thereof into the cell, the expression products of which perform a function in the transmission of signals in order to have a positive influence on signal transmission into the target cells and thereby, for example, increase the survival of T-cells.

The primary target cells for the immune therapy are T-cells of the so-called killer cell type which have a cytotoxic activity and are also referred to as TIL's (tumour infiltrating lymphocytes). The conjugates according to the invention may be used as an alternative to gene transfer using retroviral vectors in order to introduce DNA into these cells. The DNA preferably contains a gene which codes for a protein capable of increasing the cytotoxic activity of these cells, e.g. TNF or IFN-α. The DNA introduced into killer cells may also contain the IL-2 gene in order to achieve a local intensification of the proliferation of cells by the expression of IL-2.

Theoretically, all genes or gene sections which have a therapeutic or gene-therapeutic effect in cells which express a T-cell surface protein are suitable for the purposes of the present invention.

The ratio of nucleic acid to conjugate may vary within wide limits and it is not absolutely necessary to neutralise all the charges of the nucleic acid. This ratio will have to be adjusted for each individual case in accordance with criteria such as the size and structure of the nucleic acid to be transported, the size of the polycation, the number and distribution of its charges, so that there is a favourable ratio, for the particular application, between the transportability and biological activity of the nucleic acid. This ratio can initially be coursely adjusted, perhaps by means of the delay in the speed of migration of the DNA in a gel (e.g. by means of mobility shift on an agarose gel) or by density gradient centrifugation. After this preliminary ratio has been obtained it may be advisable to carry out transport tests with the radioactively labelled complex with a view to obtaining the maximum available activity of the nucleic acid in the cell and possibly reducing the conjugate portion so that the remaining negative charges of the nucleic acid do not impede transport into the cell.

The preparation of the TCBP-polycation/nucleic acid complexes may be carried out by methods known per se for the complexing of polyionic compounds. One possible way of avoiding uncontrolled aggregation or precipitation consists in mixing the two components at a high dilution (<50 μg/ml).

The TCBP-polycation-nucleic acid complexes which can be absorbed into higher eukaryotic cells by endocytosis may additionally contain one or more polycations in a non-covalently bound form which may be identical to the polycation in the conjugate, so as to increase the internalisation and/or expression of the nucleic acid achieved by means of the conjugate.

With the aid of such measures, which are the subject matter of the unpublished German Patent Application No. 41 04 186.0, a smaller amount of TCBP-polycation conjugate is required, based on the quantity of nucleic acid to be imported into the cell, to achieve at least the same efficiency of transfection/expression, which means on the one hand that synthesis is less costly. A smaller amount of conjugate may also be advantageous when it is desirable to avoid the effect of having several adjacent “docking sites” occupied by a large number of TCBP molecules within a complex, with the consequence that they are no longer available for additional complexes. Restricting the quantity of TCBP contained in the complexes to the necessary minimum, i.e. keeping the quantity of conjugate as small as possible and diluting it with free polycation, is particularly advantageous when there is only a small number of cell surface proteins on the target cells to be treated.

With the aid of such measures, the performance of conjugates which are not particularly efficient per se can be increased substantially and the performance of conjugates which are already highly efficient can be increased still further.

With regard to the qualitative composition of the complexes according to the invention, first of all the nucleic acid to be imported into the cell and the TCBP are generally determined. The nucleic acid is defined primarily by the biological effect to be achieved in the cell, e.g. by the target sequence of the gene or gene section to be inhibited or (when used in gene therapy) to be expressed, e.g. in order to substitute a defective gene. The nucleic acid may optionally be modified, e.g. because of the need for stability for the particular application.

Starting from the determination of nucleic acid and TCBP the polycation is matched to these parameters, the size of the nucleic acid being of critical importance, particularly with regard to the substantial neutralisation of the negative charges.

When choosing the non-covalently bound polycations which may be contained in the complexes, it is crucial that the addition of these substances should bring about an increase in the internalisation/expression of the nucleic acid, compared with that which can be achieved by means of the conjugates.

Like the qualitative composition, the quantitative composition of the complexes is also determined by numerous criteria which are functionally connected with one another. When deciding to provide non-convalently bound polycation as an ingredient of the complex it is crucial to determine whether and to what extent it is necessary or desirable to condense the nucleic acid, what charge the total complex should have, to what extent there is a binding and internalising capacity for the particular type of cell and to what extent it is desirable or necessary to increase it. Other parameters for the composition of the complex are the accessibility of the TCBPs for the cell surface protein, the crucial factor being the way in which this protein is presented within the complex relative to the cell. Another essential feature is the accessibility of the nucleic acid in the cell in order to perform its designated function.

The polycations contained in non-covalently bound form in the complexes may be the same as or different from those contained in the conjugate. An essential criterion for selecting them is the size of the nucleic acid, particularly with respect to the condensation thereof; with smaller nucleic acid molecules, compacting is not generally required. The choice of the polycations, in terms of the nature and quantity thereof, is also made in accordance with the conjugate, particular account being taken of the polycation contained in the conjugate: if for example the polycation is a substance which has no or very little capacity for DNA condensation, it is generally advisable, for the purpose of achieving efficient internalising of the complexes, to use those polycations which possess this quality to a greater extent. If the polycation contained in the conjugate is itself a substance which condenses nucleic acid and if adequate compacting of the nucleic acid for efficient internalisation is achieved, it is advisable to use a polycation which brings about an increase in expression by other mechanisms.

What is essential for the non-covalently bound polycation which may optionally also be contained in the complex is its ability to condense nucleic acid and/or to protect the latter from undesirable breakdown in the cell.

The invention further relates to a process for introducing nucleic acid or acids into human or animal cells which express the T-cell surface antigen, whereby a TCBP-polycation/nucleic acid complex which is preferably soluble under physiological conditions is brought into contact with the cells, particularly T-cells.

Within the scope of the present invention, the luciferase gene was used as the reporter gene to form the DNA component. (In preliminary trials with transferrin-polycation/DNA complexes in which the luciferase gene was used as a reporter gene, it was found that the efficiency of import of the luciferase gene indicates whether other nucleic acids can be used; the nucleic acid used is, in qualitative terms, not a limiting factor for the use of protein-polycation-DNA complexes.)

For certain embodiments of the present invention it may be useful to create conditions under which the degradation of the nucleic acid in the cells is inhibited or prevented.

Conditions under which the breakdown of nucleic acids is inhibited may be provided by the addition of so-called lysosomatropic substances. These substances are known to inhibit the activity of proteases and nucleases in lysosomes and are thus able to prevent the degradation of nucleic acids (Luthmann & Magnusson, 1983).

These substances include chloroquin, monensin, nigericin, ammonium chloride and methylamine.

The necessity of using a substance selected from the group of lysosomatropic substances within the scope of the invention will depend in particular on the type of cell to be treated, or if different antibodies are used, it will depend on different mechanisms by which the complexes are absorbed into the cell. Thus, for example, within the scope of the present invention, it was found that the import of DNA into the cell was differently affected by chloroquin when different antibodies were used (monoclonal anti-CD4 antibodies).

In any case, it is necessary to test the necessity for or suitability of such substances within the scope of the present invention by means of preliminary trials.

The invention further relates to pharmaceutical compositions containing as active component one or more therapeutically or gene therapeutically active nucleic acids complexed with a TCBP-polycation conjugate (TCBP-polycation conjugate and nucleic acid may also occur separately and be complexed immediately before therapeutic use).

Examples of therapeutically active nucleic acids include the antisense oligonucleotides or ribozymes mentioned hereinbefore or the genes coding for them or genes coding for transdominant mutants, which have an inhibiting effect on endogenous or exogenous genes or gene products contained in the particular target cells. These include, for example, those genes which, by virtue of their sequence specificity (complementarity to target sequences, coding for transdominant mutants (Herskowitz, 1987)), bring about an intracellular immunity (Baltimore, 1988) against HIV and can be used in the treatment of the AIDS syndrome or to prevent activation of the virus after infection.

The pharmaceutical preparations may be used to inhibit viral sequences, e.g. HIV or related retroviruses in the human or animal body. An example of therapeutic application by inhibiting a related retrovirus is the treatment of proliferative T-cell leukaemia which is caused by the HTLV-1 virus.

In addition to the treatment of viral T-cell leukaemias the present invention may also be used for treating non-viral leukaemias. Recently the involvement of oncogenes (abl, bcr, ras, rat, c-myc, N-myc) in the formation of lymphatic leukaemias has been demonstrated; it is thought probable that there are other oncogenes, on the basis of observed specific chromosome translocations. Cloning together with a knowledge of the DNA sequence of these oncogenes forms the basis for the construction of oncogene-inhibiting nucleic acid molecules and hence for a further possible therapeutic use of the present invention.

Another important field of use is gene therapy. In theory, in the scope of gene therapy by means of the present invention it is possible to use all those genes or sections thereof in target cells which express T-cell surface protein, the expression of which produces a therapeutic effect in this type of cell, e.g. by substituting genetically caused defects or by triggering an immune response.

Although the emphasis for the application of the invention has been laid on examples of cells of the T-lymphocyte lineage, it may also be laid on examples of other cell species, provided that these cells express the T-cell surface protein.

### SUMMARY OF THE FIGURES

FIG. l: Import of antiCD4-polylysine/pRSVL complexes into CD4⁺-CHO cells

FIG. 2: Import of antiCD4-polylysine/pRSVL complexes into CD4⁺-CHO cells

FIG. 3: Import of gp120-polylysine/pRSVL complexes into CD4⁺-CHO cells

FIG. 4: Import of gp120-polylysine 190/pRSVL complexes containing non-covalently bound poly(D)lysine 240 into CD4⁺-CHO cells

FIG. 5: Import of gp120-polylysine 120/RSVL complexes into CD4⁺-HeLa cells

FIG. 6: Transfer and expression of DNA in H9 cells by means of antiCD7-polylysine 190 conjugates

FIG. 7: Transfection of CD4 cells with protein A-polylysine conjugates

The invention is illustrated by means of the Examples which follow.

### EXAMPLE 1

### Preparation of AntiCD4-polylysine 90 Conjugates

Coupling was carried out analogously to methods known from the literature by introducing disulphide bridges after modification with succinimidyl-pyridyldithio-propionate (SPDP, Jung et al., 1981).

A solution of 1.7 mg of antiCD4 antibody (OKT4A, Ortho Diagnostic Systems) in 50 mM sodium phosphate buffer pH 7.8 was mixed with 11 μl of 10 mM ethanolic solution of SPDP (Pharmacia).

After 1 hour at ambient temperature the mixture was filtered through a Sephadex G 25 gel column (eluant 100 mM HEPES buffer pH 7.3), to obtain 1.4 mg of antiCD4, modified with 75 nmol of pyridyldithiopropionate groups. Poly(L)lysine 90 (average degree of polymerisation of 90 lysine groups (Sigma), fluorescent-labelled by means of FITC) was modified analogously with SPDP and brought into the form modified with free mercapto groups by treating with dithiothreitol and subsequent gel filtration. A solution of 38 nmol polylysine 90, modified with 120 nmol mercapto groups, in 0.5 ml of 20 mM sodium acetate buffer, was mixed with the above-mentioned modified antiCD4 with the exclusion of oxygen and left to stand overnight at ambient temperature. The conjugates were isolated by gel permeation chromatography (Superose 12, 500 mM guanidinium hydrochloride pH 7.3); after dialysis against 25 mM HEPES pH 7.3, corresponding conjugates were obtained consisting of 1.1 mg antiCD4 antibody modified with 11 nmol polylysine 90.

### EXAMPLE 2

### Preparation of AntiCD4-polylysine 190 Conjugates

A solution of 1.0 mg (6.25 nmol) of antiCD4 antibody (OKT4A, Ortho Diagnostic Systems) in 0.3 ml of 50 mM HEPES pH 7.8 was mixed with 37 μl of 1 mM ethanolic solution of succinimidyl-pyridyldithio-propionate (SPDP, Pharmacia). After 1 hour at ambient temperature the mixture was filtered over a Sephadex G 25 column (eluant 100 mM HEPES buffer pH 7.9), to obtain 0.85 mg (5.3 nmol) of antiCD4 modified with 30 nmol pyridyldithiopropionate groups. Poly(L)lysine190 (average degree of polymersation of 190 lysine groups (Sigma), fluorescent-labelled by means of FITC) was modified analogously with SPDP and brought into the form modified with free mercapto groups by treating with dithiothreitol and subsequent gel filtration. A solution of 7.7 nmol of polylysine 190, modified with 25 nmol of mercapto groups, in 0.13 ml of 30 mM sodium acetate buffer was mixed with the above-mentioned modified antiCD4 (in 0.5 ml of 300 mM HEPES pH 7.9) with the exclusion of oxygen and left to stand overnight at ambient temperature. The reaction mixture was adjusted to a content of about 0.6 M by the addition of 5 M NaCl. The conjugates were isolated by ion exchange chromatography (Mono S, Pharmacia, 50 mM HEPES pH 7.3, salt gradient 0.6 M to 3 M NaCl); after dialysis against 10 mM HEPES pH 7.3, corresponding conjugates were obtained consisting of 0.35 mg (2.2 nmol) of antiCD4 antibody, modified with 3.9 nmol of polylysine 190.

### EXAMPLE 3

### Preparation of gp120-polylysine 190 Conjugates

Coupling was carried out analogously to methods known from the literature, either by introducing disulphide bridges after modification with succinimidyl-pyridyldithiopropionate or by thioether linking after modification with N-hydroxysuccinimide 6-maleimidocaproate (EMCS, Sigma) (Fujiwara et al., 1981).

a) Disulphide-linked gp120-polylysine 190 conjugates:

A solution of 3 mg of recombinant gp120 (prepared by the method described by Lasky et al., 1986) in 50 mM HEPES pH 7.8 was mixed with 7 μl of 10 mM ethanolic solution of SPDP. After 1 hour at ambient temperature the mixture was filtered over a Sephadex G 25 gel column (eluant 100 mM HEPES buffer pH 7.9) to obtain 2.8 mg (23 nmol) of rgp120, modified with 67 nmol of pyridyldithiopropionate groups. A solution of 6.6 nmol of polylysine 190, fluorescent-labelled and described as above for the antiCD4 conjugates, modified with 23 nmol mercapto groups, in 120 μl of 30 mM sodium acetate was mixed with the modified rgp120, with the exclusion of oxygen, and left to stand overnight at ambient temperature. The reaction mixture was adjusted to a content of about 0.6 M by the addition of 5M NaCl. The conjugates were isolated by ion exchange chromatography (Mono S, Pharmacia, 50 mM HEPES pH 7.3, salt gradient 0.6 M to 3 M NaCl); after fractionation and dialysis against 25 mM HEPES pH 7.3, two conjugate fractions A and B were obtained, consisting of 0.33 mg of rgp120 modified with 1.3 nmol polylysine 190 (in the case of fraction A), and 0.34 mg of rgp120 modified with 3.2 nmol of polylysine 190 (fraction B).

b) Thioether-linked gp120-polylysine 190 conjugates:

A solution of 2 mg of recombinant gp120 in 0.45 ml of 100 mM HEPES pH 7.9 was mixed with 17 μl of a 10 mM solution of EMCS in dimethylformamide. After 1 hour at ambient temperature the mixture was filtered over a Sephadex 2 gel column (eluant 100 mM HEPES buffer 7.9). The product solution (1.2 ml) was then reacted, with the exclusion of oxygen, with a solution of 9.3 nmol of polylysine 190, fluorescent-labelled and modified as described above (antiCD4 conjugates) with 30 nmol mercapto groups (in 90 μl 30 mM sodium acetate pH 5.0), and left to stand overnight at ambient temperature. The reaction mixture was adjusted to a content of about 0.6 M by the addition of 5M NaCl. The conjugates were isolated by ion exchange chromatography (Mono S, Pharmacia, 50 mM HEPES pH 7.3, salt gradient 0.6 M to 3 M NaCl); after fractionation and dialysis against 25 mM HEPES pH 7.3, three conjugate fraction A, B and C were obtained, consisting of 0.40 mg of rgp120, modified with 1.9 nmol of polylysine 190 (in the case of fraction A), or 0.25 mg of rgp 120, modified with 2.5 nmol of polylysine 190 (fraction B), or 0.1 mg of rgp 120, modified with 1.6 nmol of polylysine 190 (fraction C).

### EXAMPLE 4

### Preparation of AntiCD7-polylysine 190 Conjugates

A solution of 1.3 mg of antiCD7 antibody (Immunotech) in 50 mM HEPES pH 7.9 was mixed with 49 μl of 1 mM ethanolic solution of SPDP (Pharmacia). After 1 hour at ambient temperature the mixture was filtered over a Sephadex G 25 gel column (eluant 50 mM HEPES buffer 7.9), to obtain 1.19 mg (7.5 nmol) of antiCD7, modified with 33 nmol of pyridyldithiopropionate groups. Poly(L)lysine 190, fluorescent labelled by means of FITC, was modified analogously with SPDP and brought into the form modified with free mercapto groups by treatment with dithiothreitol and subsequent gel filtration.

A solution of 11 nmol of polylysine 190, modified with 35 nmol mercapto groups, in 0.2 ml of 30 mM sodium acetate buffer was mixed with the above-mentioned modified antiCD7 (in 0.5 ml of 300 mM HEPES pH 7.9) with the exclusion of oxygen and left to stand overnight at ambient temperature. The reaction mixture was adjusted, by the addition of 5 M NaCl, to a content of about 0.6 M. The conjugate were isolated by ion exchange chromatography (Mono S, Pharmacia, 50 mM HEPES pH 7.3, salt gradient 0.6 M to 3 M NaCl); after dialysis against 10 mM HEPES pH 7.3, corresponding conjugates were obtained, consisting of 0.51 mg (3.2 nmol) of antiCD7 antibody, modified with 6.2 nmol of polylysine 190.

### EXAMPLE 5

### Preparation of Complexes of Antibody-Polycation Conjugates with DNA

The complexes were prepared by mixing dilute solutions of DNA (30 μg/ml or less in 150 mM NaCl, 20 mM HEPES pH 7.3) with the antibody-polylysine conjugates obtained in Examples 1, 2 and 4 (100 μg/ml or less). The DNA used was pRSVL plasmid DNA (De Wet et al., 1987) prepared by Triton-X lysis standard method (Maniatis, 1982) followed by CsCl/EtBr equilibrium density gradient centrifugation, decolorising with butanol-1 and dialysis against 10 mM Tris/HCl pH 7.5, 1 mM EDTA. In order to prevent precipitation of the DNA complexes, phosphate-free buffer was used (phosphates decrease the solubility of the conjugates).

### EXAMPLE 6

### Transfer and Expression of DNA in CD4⁺ CHO-cells by means of AntiCD4-polylysine 90 Conjugates

In this and the following Examples plasmid DNA containing the Photinus pyralis luciferase gene as reporter gene was used to investigate gene transfer and expression. In the Figures which show the results of the experiments, the values given for the luciferase activity relate to the activity of the entire cell sample.

CD4⁺ CHO-cells (Lasky et al., 1987) were seeded, at a rate of 5×10⁵ cells per T-25 vial, in Ham's F-12 medium (Ham, 1965) plus 10% FCS (foetal calves' serum). 18 hours later the cells were washed twice with Ham's F-12 medium without serum and incubated in this medium (5 ml) for 5 hours at 37° C.

Anti-CD4 polylysine/pRSVL complexes were prepared at final concentrations of DNA of 10 μg/500 μl in 150 mM NaCl, 20 mM HEPES pH 7.5, as described in Example 5. Anti-CD4 polylysine 90 (8.4 nmol polylysine 90/mg anti-CD4) were used in the mass ratios specified (from 1.9 to 8.1 expressed as mass of anti-CD4). In samples 1 to 4 the complexes were added to the cells in Ham's F-12 medium without serum, containing 100 μM chloroquin; in samples 5 and 6 the chloroquin was omitted. After 4 hours' incubation the cells were washed twice with medium plus 10% FCS and incubated in this medium. In samples 5 and 6 the same volume of serum-containing medium was added to the cells. After 20 hours all the cells were washed with fresh serum-containing medium and harvested 48 hours later. Aliquots of extracts (about ⅕ of each sample, corresponding to the same amount of protein, were investigated for lucerifase activity (De Wet et al., 1987). The bioluminescence was measured using clinilumate (Berthold, Wildbach, FRG). The result of these investigations is shown in FIG. 1. It was found that DNA is imported into CD4 cells by means of the conjugates according to the invention and the imported DNA is expressed, the efficiency of the DNA import being proportional to the content of anti-CD4/polylysine.

### EXAMPLE 7

### Transfer and Expression of DNA into CD4 CHO Cells by means of AntiCD4-polylysine 190 Conjugates

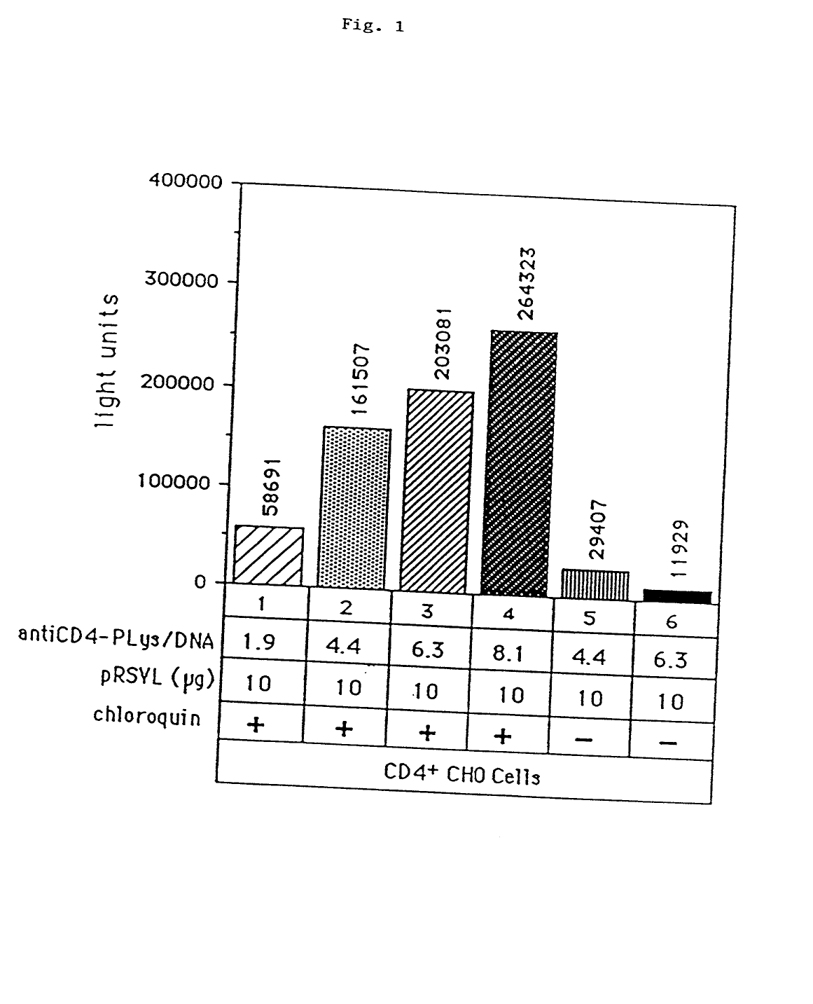

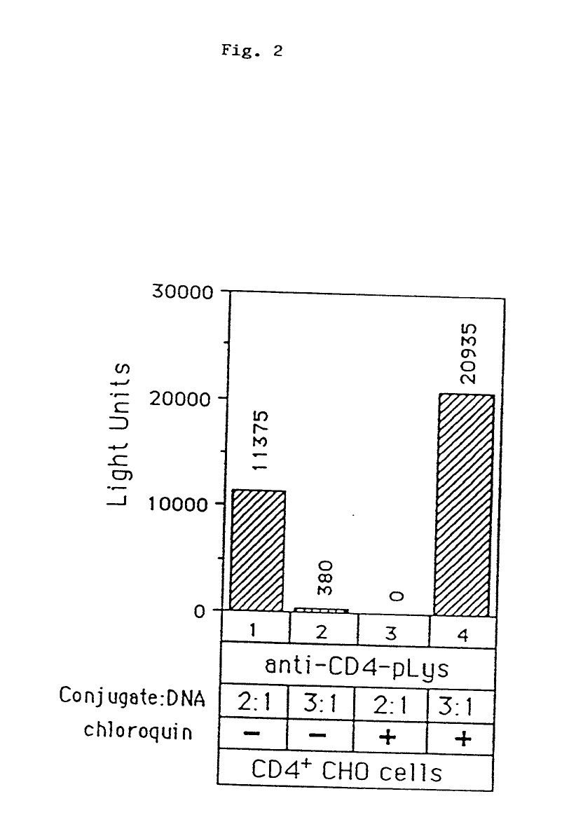

First, CD4⁺ CHO cells were cultivated as described in Example 6. Conjugate/DNA complexes, prepared as in Example 5, containing 10 μg PRSVL and either a 2:1 or 3:1 mass excess of antiCD4-polylysine 90 (see Example 1) or gp120 polylysine (see Example 3), as stated in FIG. 2, were added to the cells in the absence or presence of 100 μM chloroquin. After a further 4 hours at 37° C. the samples containing chloroquin were washed twice with Ham's medium, containing 10% foetal calves' serum, whilst 5 ml of the same medium were added to the samples containing no chloroquin. The cells were incubated for a further 20 hours at 37° C. and aliquots were investigated for their luciferase activity, as stated in Example 6. The results of these tests are shown in FIG. 2.

### EXAMPLE 8

### Import of gp120-polylysine/pRSVL Complexes into CD4⁺ CHO Cells

a) Preparation of gp120-polylysine/DNA Complexes

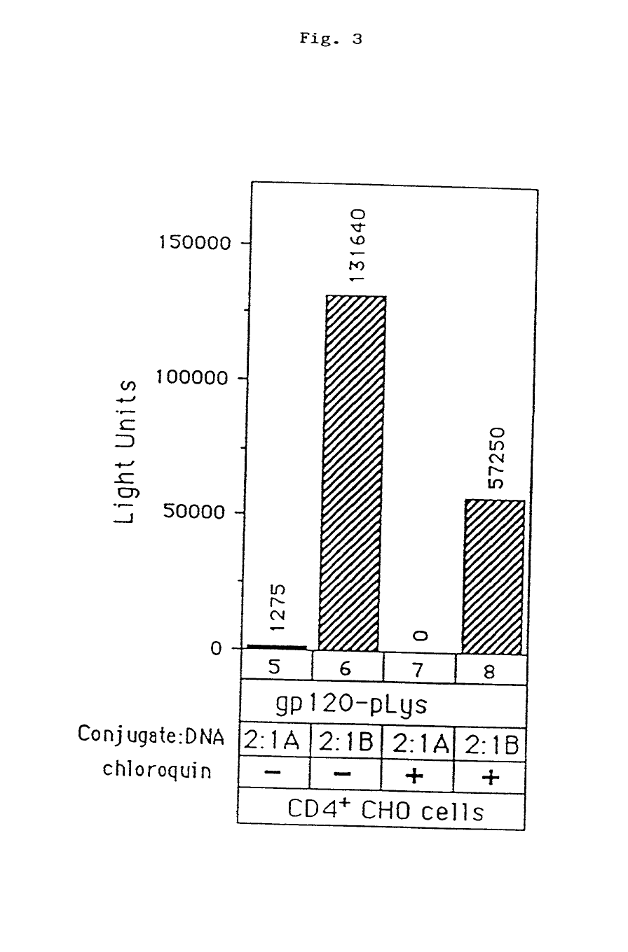

The complexes were prepared by first diluting 6 μg of DNA in 330 μl of HBS at ambient temperature (100 μg/ml or less). The DNA used was pRSVL plasmid DNA (cf. Example 5). Aliquots of the gp120-pL190 conjugates contained in Example 3 (in amounts specified in FIG. 3) were diluted in 170 μl HBS. The conjugate dilution in each case was quickly added to the DNA dilution, incubated for 30 minutes and then used for transfection.

b) Transfection of CD⁴⁺ cells

CD4 CHO cells (Lasky et al., 1987) were seeded out, at the rate of 6×10⁵ cells per T-25 vial, in Ham's F-12 medium (Ham, 1965) plus 10% FCS (foetal calves' serum).

18 hours later the cells were washed twice with Ham's F-12 medium without serum and incubated in this medium (5 ml) for 5 hours at 37° C. Then the solutions of the gp120-pL/pRSVL complexes were added to the cells. After 4 hours an equal volume of DME medium (Dulbecco's modified Eagle's medium) containing 10% foetal calves' serum was added to each sample. After 24 hours the cells were harvested, extracts were prepared and aliquots of similar protein content (about ⅕ of the total material) were investigated for luciferase activity as in the previous Examples. The values given in FIG. 3 correspond to the luciferase activity of 6×10⁵ cells. It was found that the activity of the gp120-pL conjugates depends on the ratio of components, the greater activity being found in the conjugates having a low gp120:polylysine ratio (fraction C, traces 5 and 6) whilst a very low or no activity was found in the fraction having a high gp120:polylysine ratio (fraction A, traces 1 and 2).

### EXAMPLE 9

### Import of gp120-polylysine/pRSVL Complexes Containing Non-covalently Bound Polylysine, into CD4⁺ CHO Cells

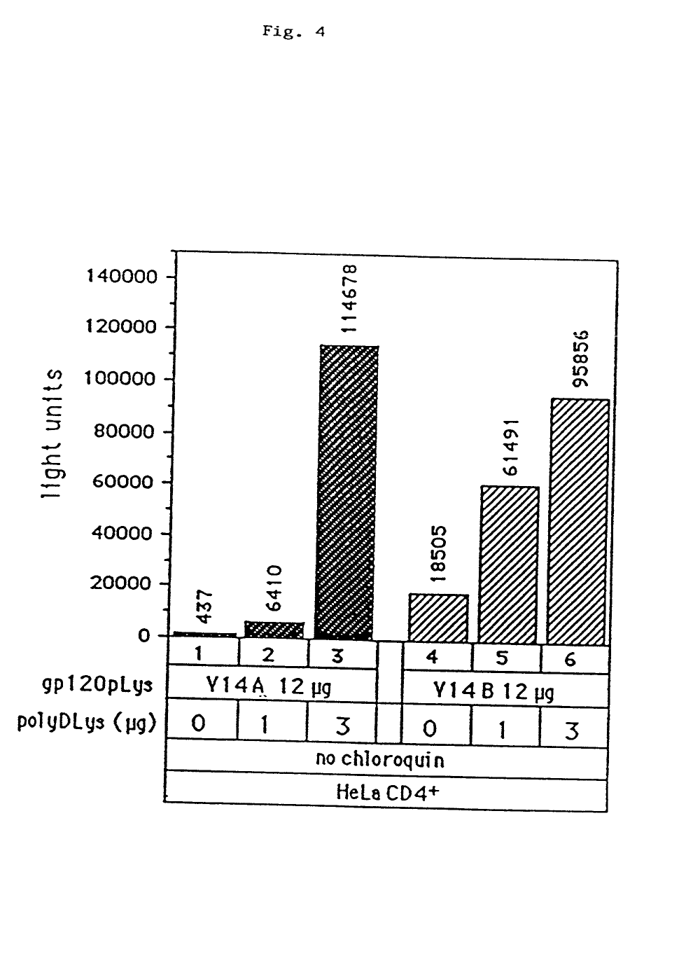

The gp120-pL conjugates which showed poor results for transfection in Example 8 (fractions A and B) were investigated to see whether the addition of free polylysine would improve the uptake of DNA. 6 μg of DNA and 12 μg of conjugate were used, 1 or 3 μg of polylysine 240 being added to the conjugate before the complexing with DNA. In accordance with the results obtained for the transferrin conjugates, a sharp increase in the luciferase activity was observed (260-fold and 5.2-fold, respectively) (FIG. 4).

### EXAMPLE 10

### Import of gp120-polylysine/pRSVL Complexes into CD4⁺ HeLa Cells

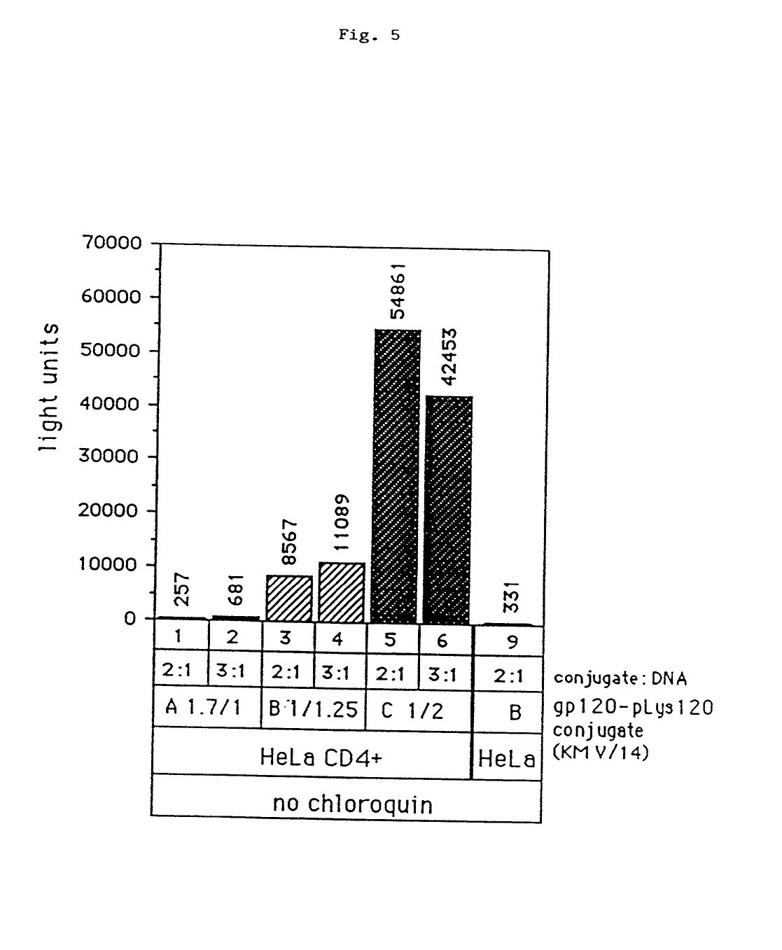

CD4⁺ HeLa cells (Maddon et al., 1986) or normal HeLa cells as the control in DME medium plus 10% FCS were seeded out at the rate of 6×10⁵ cells per T25 vial and then cultivated as described in Example 6 for CHO-cells. The cells were brought into contact with gp120-polylysine/pRSVL complexes in the specified ratios of conjugate to DNA (FIG. 5) (the gp120-polylysine conjugates A, B and C being three fractions of a Mono S separation of the conjugated material of Example 3b)). The molar ratio of gp120 to polylysine of each fraction is given in the Figure. After 4 hours contact with the conjugates in the absence of serum, serum-containing medium was added and the cells were harvested after 20 hours. From the cell extracts, aliquots standardised for identical protein content were examined for their luciferase activity. The values given in the Figure correspond to the luciferase activity of 6×10⁵ cells transfected with 6 μg DNA.

### EXAMPLE 11

### Transfer and Expression of DNA in H9-cells by Means of AntiCD7-polylysine 190 Conjugates

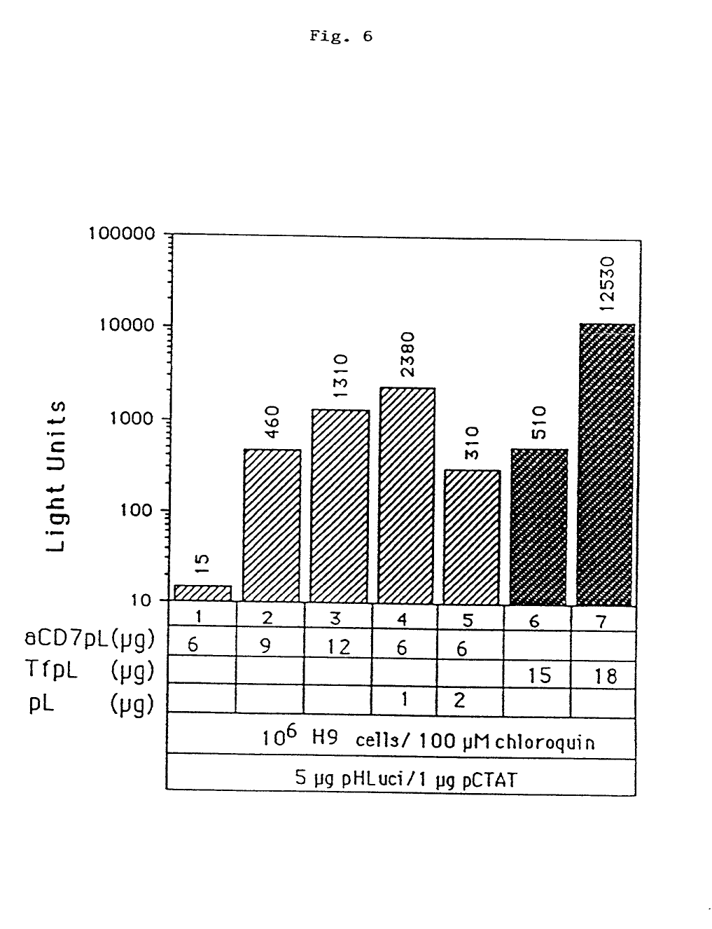

a) Cells of the T-cell line H9 (Mann et al., 1989) were cultivated in RPMI 1640 medium, supplemented with 20% FCS, 100 units per ml of penicillin, 100 μg/ml of streptomycin and 1 mM glutamine. Immediately before transfection the cells were collected by centrifuging and taken up in fresh medium at the rate of 100,000 cells per ml (1,000,000 cells per sample), which were used for transfection. As a comparison with antiCD7 conjugates, transferrin conjugates were used. Transferrin-polylysine conjugates were prepared as described in EP-A 1 388 758; the antiCD7 conjugates used were those described in Example 4. Complexing with DNA was carried out as stated in Example 5. For transient transfection in H9 cells the DNA used was the plasmid pHLuci which contains the HIV-LTR sequence combined with the sequence which codes for luciferase, followed by the SV40-intron/polyA site: the HindIII fragment containing the protease 2A gene from pHIV/2A (Sun and Baltimore, 1989) was removed and replaced by a HindIII/SmaI fragment of pRSVL (De Wet et al., 1987) containing the sequence which codes for luciferase. The two fragments were joined via the HindIII sites (after smooth ends had been produced using Klenow fragment) and then linked via the smooth SmaI site to the now smooth HindIII site. A clone having the correct orientation of the luciferase gene sequence was selected. This plasmid requires the TAT gene product for a strong transcription activity. This is prepared by co-transfection with the plasmid pCMVTAT, which codes for the HIV-TAT gene under the control of the CMV immediate early promoter (Jakobovits et al., 1990). The DNA complexes used for transfection contain a mixture of 5 μg of pHLuci and 1 μg of pCMVTAT. The DNA/polycation complexes (500 μl) were added to the 10 ml cell sample and incubated for 4 hours in the presence of 100 μM chloroquin. Then the cells were washed in fresh medium, harvested 40 hours later and investigated for their luciferase activity as described in the preceding Examples. The results (in luciferase light units) are given in FIG. 6: it was found that the luciferase activity increases as the amount of antiCD7-polylysine conjugate complexed with 6 μg of DNA increases (samples 1, 2 and 3). A further increase in activity was observed when 6 μg of conjugate were used together with 1 μg of free polylysine for complex formation (sample 4), whilst a further addition of polylysine affected the gene transport (sample 5). (The comparison tests carried out with transferrin-polylysine conjugates are designated 6 and 7.)

b) A further series of tests for transfection using the antibody conjugates was carried out using the plasmid pSSTNEO. This plasmid, which contains a neomycin resistance gene as marker, was introduced into H9 cells using antiCD4, antiCD7 and (for comparison) transferrin-polylysine 190 conjugates (6 μg of DNA were used per 10⁶ cells; the optimum transfection conditions had been determined in preliminary trials using transient luciferase assays). The plasmid pSSTNEO contains the large Sst fragment of the pUCu locus (Collis et al., 1990) which contains the HSV TK-neo unit. A 63 bp fragment containing a single NdeI site had been introduced into the Asp718 site. Aliquots of the transfected cells (containing a defined number of cells) were then diluted in a semisolid methylcellulose medium containing 1000 μg/ml G418. In order to do this, aliquots of the cells were plated out 3 days after transfection with DNA, containing the neomycin marker, in a semisolid medium which contained in addition to the normal requirements 0.5-1 mg/ml of G418 and 20 mg/ml of methylcellulose. (In order to prepare the semisolid selection medium a solution of 20 g of methylcellulose in 460 ml of water was prepared under sterile conditions.) Then 500 ml of doubly concentrated, supplemented nutrient medium, also prepared under sterile conditions, were combined with the methylcellulose solution, the volume was adjusted to 1 litre and the medium was stirred overnight at 4° C. 50 ml aliquots of this medium were mixed with 10 ml of serum, optionally after storage at −20° C., and the volume was adjusted to 100 ml with complete medium containing no serum. At this stage G418 was added. A 2.5 ml aliquot of the methylcellulose medium was mixed with a 50 to 100 μl aliquot of the cell suspension and about 1 ml of this mixture was poured into culture dishes. Incubation was carried out at 37° C. under a CO₂ atmosphere. About 10 to 14 days later the G418-resistant cells were counted (only colonies containing more than 200 cells were counted as positive). The results are shown in FIG. 4 (this shows the number of G418-resistant colonies per 1000 cells 10 days after being placed in the antibiotic medium).

### EXAMPLE 12

### Preparation of Protein-A Polylysine 190 Conjugates

A solution of 4.5 mg of protein-A (Pierce, No. 21182, 107 nmol) in 0.5 ml of 100 mM HEPES pH 7.9 was mixed with 30 μl of 10 mM ethanolic solution of SPDP (Pharmacia). After 2 hours at ambient temperature the mixture was filtered over a Sephadex G25 gel column (eluant 50 mM HEPES buffer pH 7.9) to obtain 3.95 mg (94 nmol) of protein-A, modified with 245 nmol of pyridyldithiopropionate groups. Poly(L)lysine 190, fluorescent-labelled by means of FITC, was modified analogously with SPDP and, by treatment with dithiothreitol and subsequent gel filtration, brought into the form modified with free mercapto groups. A solution of 53 nmol of polylysine 190, modified with 150 nmol of mercapto groups, in 0.8 ml of 30 mM sodium acetate buffer was mixed with the above-mentioned modified protein-A, under the exclusion of oxygen, and left to stand overnight at ambient temperature. The reaction mixture was adjusted to a content of approximately 0.6 M by the addition of 5 M NaCl. The conjugates were isolated by ion exchange chromatography (Mono S, Pharmacia, 50 mM HEPES pH 7.3, salt gradient 0.6 M to 3 M NaCl); after fractionation and dialysis against 25 mM HEPES pH 7.3, two conjugate fractions A and B were obtained, consisting of 1.15 mg (27 nmol) of protein A, modified with 5 nmol of polylysine 190 (in the case of fraction A) and 2.6 mg (6.2 nmol) of protein-A, modified with 40 nmol of polylysine 190 (fraction B). The complexes with DNA were prepared analogously to Example 5.

### EXAMPLE 13

### Transfection of CD4⁺ Cells with Protein A-polylysine Conjugates

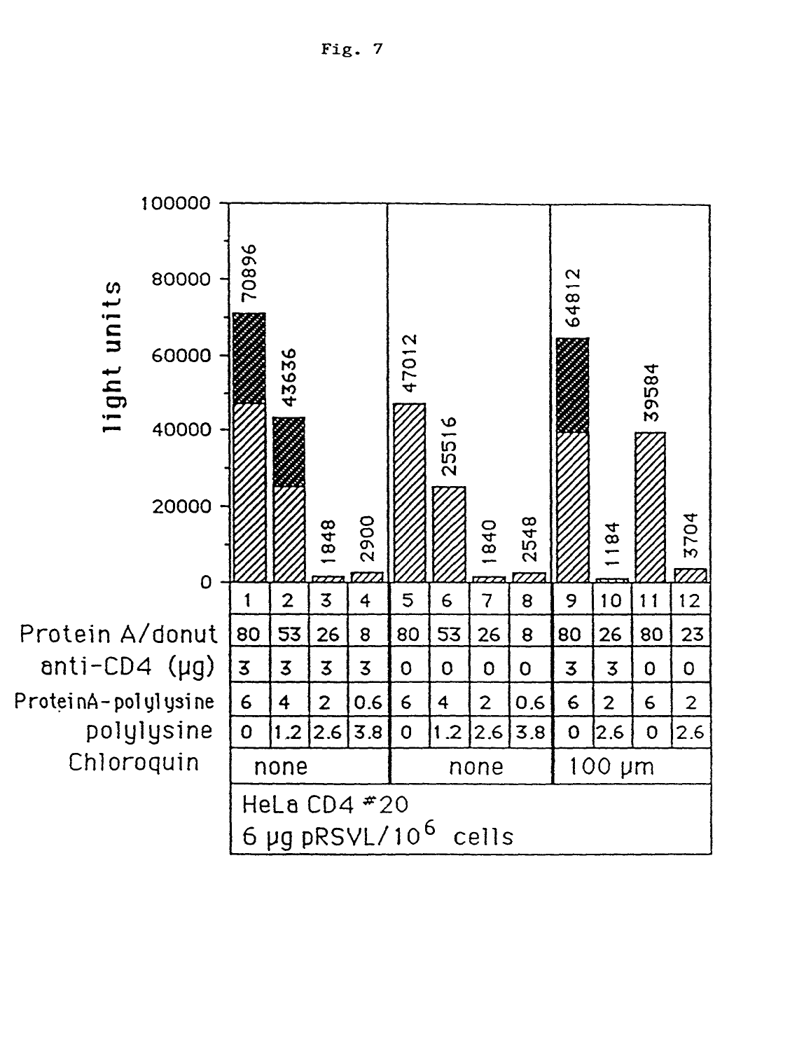

CD4-expressing HeLa cells (see Example 10) were seeded out at the rate of 6×10⁵ cells per T25 vial and then grown in DME medium plus 10% FCS. Where shown in FIG. 7, the cells were pre-incubated with the antibody (anti-CD4gp55kD, IOT4, Immunotech) (3 μg per sample) for 1 hour at ambient temperature. In the meantime, protein A polylysine 190/DNA complexes were prepared as in Example 5 in 500 μl of HBS, containing 6 pg of pRSVL and the specified amounts of protein A-polylysine 190 plus additional free polylysine. At the end of the 1 hour incubation the cells were placed in 4.5 μl of fresh medium and the 500 μl DNA sample was added to the cell at 37° C. After 4 hours those samples which contained 100 μM chloroquin (samples 9-12) were washed in fresh medium, whilst samples 1-8 were incubated until harvesting with the DNA. For the luciferase assay the cells were harvested 20 hours later. The results of the tests are shown in FIG. 7. It was found that the luciferase activity was dependent on the presence of protein A-polylysine in the DNA complex (samples 1-4, 5, 6). In samples 5-8, 11, 11, 12, DNA transport by means of the protein A complex was demonstrated without any antibody pretreatment; however, the DNA import was increased by about 30% if the cells had been pretreated with the antibody which recognises the cell surface protein CD4 (samples 1-4, 9, 10). It was also found that the presence of chloroquin does not cause any increase in DNA expression (cf. samples 1-8 with samples 9-12).

Bibliography:

Aruffo A. and Seed B., 1987, EMBO J. 6, No. 11, 3313-3316

Baltimore D., 1988, Nature 335, 395

Chaudhary V. K. et al., 1990, Proc.Natl.Sci.USA 87,

Collis P. et al., 1990, EMBO J. 9, 233-240.

Dalgleish A. G. et al., 1984, Nature 312, 763

De Wet et al., 1987, Mol.Cell.Biol. 7, 725-737

Fauci A., 1988, Science 239, 617

Felsenfeld et al., 1978, Nature 271, 115

Fujiwara et al., 1981, J.Immunol.Meth. 45, 195

Green M. et al., 1989, Cell 58, 215-223

Ham R. G., 1965, Proc.Natl.Acad.Sci.USA 53, 288

Herskowitz I., 1987, Nature 329, 219

Jakobovits A. et al., 1990, EMBO J. 9, 1165-1170

Johnston S. A., 1990, Nature 346, 776-777

Jung et al., 1981, Biochem.Res.Commun.101, 599

Kasid A. et al., 1990, Proc.Natl.Acad.Sci.USA 87, 473-477

Klatzmann D. et al., 1984, Nature 312, 767

Lasky et al., 1986, Science 233, 209-212

Lasky et al., 1987, Cell 50, 975-985

Luthmann and Magnussen, 1983, Nucl.Acids Res.11 1295-1308

Maddon P. J. et al., 1986, Cell 47, 333-348

Malim M. et al., 1989, Cell 58, 205-214

Maniatis, 1982, Molecular Cloning, Cold Spring Harbor

Mann D. L. et al., 1989, AIDS Research and Human Retroviruses, 5, 253

McDougal J. S. et al., 1986, Science 231, 382

Nabel E. G. et al., 1990, Science 249, 1285-1288

Nygren A. et al., 1988, Proc.Natl.Acad.Sci.USA 85, 6543-6546

Orlandi R. et al., 1989, Proc.Natl.Acad.Sci.USA 86, 3833-3837

Pauza C. D. and Price T. M., 1988, J.Cell Biol. 107, 959-968

Pelchen-Matthews et al., 1989, EMBO J.8, 3641-3649

Sastry L. et al., 1989, Proc.Natl.Acad.Sci.USA 86, 5728-5732

Smith D. H. et al., 1987, Science 238, 1704-1707

Stein B. S. et al., 1987, Cell 49, 659

Surolia A. et al., 1982, TIBS 7, 74-75

Trono D. et al., 1989, Cell 59, 113-120

Warrant R. et al., 1978, Nature 271, 130-135

Wilson J. M. et al., 1990, Proc.Natl.Acad.Sci.USA 87, 439-443

Wu G. Y. and Wu C. H., 1987, J.Biol.Chem. 263, 14621-14624

Yang N. S. et al., 1990, Proc.Natl.Acad.Sci.USA 87, 9568-9572

Zamecnik et al., 1986, Proc.Natl.Acad.Sci.USA 83, 4143

Zon G., 1988, Pharmaceut. Research 5, No.9, 539-549

1. The protein-polycation conjugates which are capable of forming, with nucleic acids or nucleic acid analogues, soluble complexes which are absorbed into human or animal cells, characterised in that the protein component of the conjugates is a protein capable of binding to a cell surface protein expressed by cells of the T-cell lineage, so that the complexes formed are taken up into cells which express the T-cell surface protein.

2. Conjugates according to claim 1,characterised in that their protein component is a preferably monoclonal antibody or a fragment thereof, directed against the T-cell surface protein.

3. Conjugates according to claim 1 or 2, characterised in that they contain a protein capable of binding to CD4.

4. Conjugates according to claim 3 characterised in that they contain a monoclonal anti-CD4 antibody or the fragment thereof which contains a gp120 binding epitope.

5. Conjugates according to claim 3, characterised in that they contain as protein HIV-1 gp120 or a homologous protein of related retroviruses or a fragment thereof which binds to CD4.

6. Conjugates according to claim 1 or 2, characterised in that they contain a protein which binds to a tumour marker expressed on T-cells.

7. Conjugates according to claim 6, characterised in that they contain a protein which binds to CD7.

8. Conjugates according to one of claims 2, 4, 6 or 7, characterised in that they contain an antibody in a form which is directly coupled to the polycation.

9. Conjugates according to one of claims 2, 4, 6 or 7, characterised in that they contain an antibody in a form bound by means of a protein A coupled to polycation.

10. Protein A-polycation conjugates for preparing antibody conjugates according to claim 9.

11. Conjugates according to claim 1, characterised in that the polycation is an optionally modified protamine.

12. Conjugates according to claim 1, characterised in that the polycation is an optionally modified histone.

13. Conjugates according to claim 1, characterised in that the polycation is a synthetic homologous or heterologous polypeptide.

14. Conjugates according to claim 13, characterised in that the polypeptide is polylysine.

15. Conjugates according to one of claims 11 to 14, characterised in that the polycation has about 20 to 500 positive charges.

16. Conjugates according to one of claims 11 to 15, characterised in that the molar ratio of T-cell binding protein to polycation is about 10:1 to 1:10.

17. New protein-polycation/nucleic acid complexes which are absorbed into human or animal cells, characterised in that the protein component of the conjugates is a protein capable of binding to a cell surface protein expressed by cells of the T-cell lineage, so that the complexes formed are taken up in cells which express the T-cell surface protein.

18. Complexes according to claim 17, characterised in that they contain as the conjugate component one of the conjugates defined in claims 1 to 9 or 11 to 16.

19. Complexes according to one of claims 17 or 18, characterised in that they additionally contain a non-covalently bound polycation, which may optionally be identical to the polycation of the conjugate, so that the internalisation and/or expression of the nucleic acid achieved by the conjugate is increased.

20. Complexes according to one of claims 17 to 19, characterised in that they contain a virus inhibiting nucleic acid.

21. Complexes according to claim 20, characterised in that they contain a nucleic acid which inhibits replication and expression of the HIV-1 virus or related retroviruses.

22. Complexes according to claim 21, characterised in that the inhibiting nucleic acid is complementary to sequences of the HIV-1 genome.

23. Complexes according to claim 22, characterised in that the nucleic acid is complementary to sequences of the tat gene.

24. Complexes according to claim 22, characterised in that the nucleic acid is complementary to sequences of the rev gene.

25. Complexes according to claim 22, characterised in that the nucleic acid is complementary to sequences of the nef gene.

26. Complexes according to claim 22, characterised in that the nucleic acid is complementary to LTR-sequences.

27. Complexes according to claim 22, characterised in that the nucleic acid is complementary to the tar sequence.

28. Complexes according to one of claims 20 to 27, characterised in that they contain an inhibiting nucleic acid in the form of a ribozyme, optionally together with a carrier RNA, or the gene coding therefor.

29. Complexes according to claim 28, characterised in that they contain a nucleic acid in the form of a genetic unit consisting of a tRNA-gene as carrier gene and a ribozyme gene arranged within this gene.

30. Complexes according to one of claims 20 to 27, characterised in that they contain an inhibiting nucleic acid in the form of an optionally modified antisense oligonucleotide, optionally together with a carrier nucleic acid, or in the case of an RNA-oligonucleotide, the gene coding therefor.

31. Complexes according to claim 20, characterised in that they contain a nucleic acid coding for a virus protein which contains a trans-dominant mutation.

32. Complexes according to one of claims 17 to 19, characterised in that they contain an oncogene-inhibiting nucleic acid.

33. Complexes according to claim 32, characterised in that they contain an oncogene-inhibiting nucleic acid in the form of a ribozyme, optionally together with a carrier RNA or the gene coding therefor.

34. Complexes according to claim 33, characterised in that they contain an oncogene-inhibiting nucleic acid in the form of a ribozyme, optionally together with a carrier RNA, or the gene coding therefor.

35. Complexes according to one of claims of 17 to 19, characterised in that they contain as nucleic acid a therapeutically or gene therapeutically active gene or gene section.

36. Process for introducing nucleic acid or acids into cells which express a T-cell surface protein by forming one of the complexes defined in claims 17 to 35, which is preferably soluble under physiological conditions, from one of the protein-polycation conjugates defined in claims 1 to 9 or 11 to 16 and nucleic acid or acids, optionally in the presence of non-covalently bound polycation, and bringing cells which express the T-cell surface protein, especially T-cells, into contact with this complex, optionally under conditions under which the breakdown of nucleic acid in the cell is inhibited.

37. Process according to claim 36, in which a complex is formed from a protein A-polycation conjugate, consisting of protein A and one of the polycations defined in claims 11 to 15 and one of the nucleic acids defined in claims 20 to 35, and the complex is brought into contact, in the presence of an antibody directed against a T-cell surface protein, with cells which express this surface protein, the antibody being bound to the conjugate component of the complex.

38. Pharmaceutical preparation containing as active component one or more therapeutically or gene therapeutically active nucleic acids in the form of one of the complexes defined in claims 17 to 35.
